(19) United States
(12) Patent Application Publication
(10) Pub. No.: US 2024/0404662 A1
(43) Pub. Date: Dec. 5, 2024

(54) SUPPORT SYSTEM

(71) Applicant: Drägerwerk AG & Co. KGaA, Lübeck (DE)

(72) Inventors: Frank FRANZ, Lübeck (DE); Birger LANDWEHR, Lübeck (DE)

(21) Appl. No.: 18/678,066

(22) Filed: May 30, 2024

(30) Foreign Application Priority Data
Jun. 1, 2023 (DE) 10 2023 114 373.5

(51) Int. Cl. G16H 10/60; G16H 50/20; G16H 50/30

(52) U.S. Cl. CPC G16H 10/60; G16H 50/20; G16H 50/30

(57) ABSTRACT

A support system (1) supports (assists) a patient (3000) during the performance of medical diagnostics or therapy. The support system (1) includes at least one interface (3), a control unit (5), a data analysis unit (6), a data storage unit (7) and an output unit (9). Instructions (8), instruction groups or instruction families (88) are stored in the data storage unit (7). The instructions are selected by the control unit (5) on the basis of situations of the patient (3000) during therapies, diagnoses, care measures and are transmitted to the patient (3000) with the output unit (9).

### CROSS REFERENCE TO RELATED APPLICATIONS

This application claims the benefit of priority under 35 U.S.C. § 119 of German Application 10 2023 114 373.5, filed Jun. 1, 2023, the entire contents of which are incorporated herein by reference.

### TECHNICAL FIELD

The present invention relates to a support system (assistive system) for a patient.

### BACKGROUND

A support system for a patient enables the clinical staff to accompany and support the patient during the patient's stay in healthcare facilities during diagnostic, therapeutic or nursing activities or measures. The support system enables the patient to be addressed or messaged (spoken to) in the course of care, diagnosis or therapy, as well as in preparation for activities or measures intended by the clinical staff.

In particular embodiments of the support system, various aspects of human-machine communication with the patient can be trained at different communication levels or combinations of communication levels, which take place via the patient's eyes, ears, nose and skin.

In special configurations of the support system, various aspects can be trained to determine and evaluate the patient's participation in human-machine communication.

In special configurations of the support system, various aspects of an evaluated or weighted inclusion of the patient's participation in human-machine communication can be trained.

The state of the art provides ways of identifying situations or events that arise in connection with the care or treatment of a patient. For example, DE102016013138 A1 shows an indication of the need to perform suction on a patient.

US2021225489 AA shows—based on facial expression and movement data—a way to determine whether a patient is likely to extubate himself/herself.

U.S. Pat. No. 9,728,061 BB describes a way of using patient movements to identify situations in which a patient leaves the bed independently.

The state of the art also provides possibilities for detecting situations underlying mechanical ventilation (mechanical respiration). For example, EP3917379 A1 shows a detection of breathing patterns, EP2989978 B1 a detection of pathological breathing patterns, EP4003154 A1 a detection of breathing disorders, EP2830484 B1 a detection of sleep phases and waking phases, DE102016007336 A1 and DE102016013140 A1 possibilities for detecting coughing or coughing attacks.

### SUMMARY

It is an object of the invention to provide a support system which makes it possible to support a patient during his/her stay in healthcare facilities.

A further object of the present invention is to provide a process for supporting a patient in his/her diagnosis and therapy or for operating a support system.

A further object of the present invention is to provide a computer program or a computer program product (each provided with a tangible, non-transitory computer-readable medium) which enables a patient to be supported in his/her diagnosis and therapy.

These and other problems are solved with features according to the invention.

The problem of specifying a support system is solved with features of the support system according to the invention.

The problem for a process for supporting a patient in his/her diagnosis and therapy or for operating a support system is solved with features of a process for supporting a patient according to the invention.

Furthermore, the process may also be provided as a computer program or a computer program product (each provided with a tangible, non-transitory computer-readable medium) having features according to the invention, so that the scope of protection of the present application also extends to the computer program product and the computer program.

Features and details which are described in connection with the process according to the invention naturally also apply in connection with and with regard to the device and vice versa, so that with regard to the disclosure of the individual aspects of the invention, reference is or can always be made reciprocally.

Advantageous embodiments of the invention are disclosed herein and are explained in more detail in the following description with partial reference to the figures. Features and details which are described in connection with the support system according to the invention naturally also apply in connection with the process according to the invention for supporting a patient in his diagnosis and therapy and vice versa in each case, so that reference is or can always be made to the individual aspects of the invention reciprocally with regard to the disclosure.

According to the invention, the support system is configured to enable a patient to be addressed or messaged during diagnostic, therapeutic or nursing activities or measures. The support system is also configured to record and evaluate the patient's reactions to being addressed or spoken to.

The basic idea behind the invention is to involve the patient more closely in communication compared to the current clinical situation in order to promote faster recovery. Communication via the support system expands the possibilities of the medical devices used for therapy and diagnostics, for example in intensive care units or perioperative areas.

With the help of data elements provided to the support system by a ventilator (respirator), for example, patient measurements and the device status can be evaluated and, based on this, verbal instructions can then be selected, which are delivered to the patient acoustically via auditory aids (e.g. loudspeakers, headphones or bonephones (bone conduction headphones)). Natural noises and sounds can also be played to ensure that the patient feels safe and secure in their environment and is motivated to actively participate in achieving the therapy goals, thereby shortening the treatment time.

As an introduction to the topic, a particularly easy-to-understand and illustrative example of a targeted address or message to a patient based on the physiology of breathing and/or ventilation on a ventilator can be given. From an analysis of physiological data—in this case using respiratory physiological parameters, at least such as respiratory minute volume and/or respiratory rate—critical situations can be recognized and predicted in advance, which would be indicated on the ventilator at a later time by corresponding alarms resulting from monitoring threshold values in relation to the tidal volume. In some cases, these alarms can be avoided by addressing (messaging) the patient, for example by asking them to breathe in and out more deeply. If the patient cooperates with a certain degree of willingness to participate and responds well to address (message), the patient themselves can be actively involved in reducing the frequency of alarms for alarmable events and increase their own comfort with therapy, in this case ventilation.

The support system can generate acoustic output in the form of instructions such as voice prompts, sounds and noises based on the patient's current condition and the therapy and transmit them to the patient. In the form of a feedback system, the patient's willingness to participate and, in particular, specific reactions to the instructions can be evaluated in order to determine prioritizations and adjustments of possible subsequent instructions. The patient's reactions can, for example, indicate a willingness to participate or cooperate, a state of health (physical situation, physiological situation, pathological situation) or even a state of mind (psychological situation) of the patient. The support system can include prioritizations when addressing (messaging).

### Explanations and Definitions

In the context of the present invention, a large number of terms are used in the description, figures and patent claims, which are explained and/or defined in more detail below.
instructions, group of instructions, elements of instructions, intentions of instructions, character of an instruction;
confirmation address (feedback address), group of confirmation addresses;
data elements, group of data elements;
parameters of the upper airways (respiratory tract), lower airways (respiratory tract) or lungs, respiratory physiology parameters, ventilation parameters;
physical situations, psychological situations, physiological situations;
neurological parameters;
physical parameters;
parameters of the cardiovascular system or the blood circulation, vascular or lymphatic system;
therapy or treatment environment;
alarm situations;
clinical picture, diagnosis, pathological situations;
situational events during the routine of medical therapies, medical diagnosis or medical care measures and/or in the daily clinical routine;
situations involving changes in the patient's location or disconnections;
measured values and/or metrologically determined data or information;
invasive measured values, non-invasive measured values;
image data, video data, audio data.

For the purposes of the present invention, a data element or data elements means information, measured values, status data, events, which can be received, sent, processed, stored or deleted as data in the form of data elements—preferably mostly via and with the aid of an interface arrangement comprising one or more interfaces. The data elements are made available to the support system using data lines or interfaces.

For the purposes of the present invention, metrologically determined data or information means data or information which can be qualitatively and/or qualitatively detected by means of a sensor system and which indicate chemical, biological or physical states.

In the context of the present invention, a clinical picture or diagnosis is understood to mean any information that indicates a patient's state of health, for example on the basis of a medical examination or findings (diagnosis).

For the purposes of the present invention, a therapy or treatment environment is in principle to be understood as any clinical or medical environment of the patient in which an examination, diagnosis, treatment, therapy or nursing measure is carried out on the patient or with the patient.

In this therapy or treatment environment, both manual and automated treatment or therapy measures can be carried out on the patient. Possible automated therapy measures include non-invasive blood pressure measurement (NiBP) using a blood pressure cuff, automatic changes to the ventilation mode or pattern, automated processes for weaning the patient from ventilation, breathing maneuvers as well as maneuvers for secretion suctioning, automated feeds or changes in the patient's breathing position. Medication of the patient with agents for sedation, circulatory support as well as automated position changes of the patient in or with the bed.

For the purposes of the present invention, invasive measured values are to be understood as measured values which can be measured in or on the human body. These include, for example, metrological recordings in the blood circulation, such as invasive blood pressure determined by means of an invasive measuring probe or catheter in blood vessels, as well as measured values which can be obtained, for example, by invasive sampling or removal of body tissue, body fluids such as blood, lymph, urine, sweat, saliva, fluids from the brain, stomach, gall bladder, pancreas in or on the human body.

For the purposes of the present invention, non-invasive measured values are to be understood as measured values which can be measured on the human body. These include, for example, metrological recordings of body temperatures, skin temperatures, a non-invasive blood pressure determined by means of a blood pressure cuff, measurement data from spirometry, measurement curves recorded by means of electrodes, such as an electrocardiogram (ECG) or an electromyogram (EMG), an electroencephalogram (EEG), as well as the acquisition of measurement data or images from devices for ultrasound imaging (US) or electroimpedance tomography (EIT), magnetic resonance imaging (MRI), computed tomography (CT), echocardiography devices, X-ray devices (X-Ray), mammography devices, heartbeat contractions recorders (CTG).

For the purposes of the present invention, alarm situations are to be understood as different situations of mostly acoustic or visual alarms from medical devices, which are caused, for example, by falling below or exceeding threshold values. In addition, alarm situations are also to be understood as alarms activated manually by the patient or clinical staff.

For the purposes of the present invention, image data, video data and audio data are to be understood as any data or elements of data which are suitable for representing auditory information or visual information as images or image sequences.

For the purposes of the present invention, psychological situations are to be understood as situations of a patient which represent a current psychological state of the patient, for example fear, dreams, panic, confusion, moods, joy, sadness, restlessness, nervousness, memory or concentration disorders.

In the context of the present invention, pathological situations are to be understood as specific or non-specific clinical pictures (disease patterns) as well as health impairments of a patient.

For the purposes of the present invention, physiological situations are situations that represent a current physiological state of the patient, for example states of organs such as the brain, heart, lungs, liver, stomach, intestines, pancreas, kidneys, eyes, ears, nose, muscles, skeleton, nervous system and sensory organs. A physiological condition can also represent the fitness of the patient and/or the organ. Physiological states of the organs can, for example, be described by blood flow (perfusion). Physiological states of the lungs can be described, for example, by parameters such as lung volume, functional residual capacity (FRC), minute volume, tidal volume as well as by flow or pressure conditions in the lungs, furthermore also by blood flow (perfusion) or ventilation as well as a so-called V/Q index, which indicates the ventilation-to-perfusion ratio.

For the purposes of the present invention, physical situations are to be understood as different situations which represent a current physical condition or physical health of the patient, for example conditions under various physical complaints, such as breathing difficulties, headaches, limb, shoulder, back or neck complaints, cardiovascular complaints or gastrointestinal complaints.

For the purposes of the present invention, situational events during routine medical therapies are to be understood as any performance of treatment activities with the patient or on the patient. For the purposes of the present invention, situational events during routine medical diagnosis are to be understood as any performance of examination activities on the patient.

Situations with patient disconnections include, for example, situations in which the patient has a disconnection, i.e. an interruption of one or more of the patient's access lines for the supply or continuation of fluids or gas volumes, such as invasive arterial or venous accesses, feeding tubes, endotracheal tubes, nasal breathing masks, nasal cannulas (prongs), tracheostoma, intracranial pressure probes, drains or disconnections of connection lines of one or more measuring electrodes of EIT, ECG, EMG or EEG.

Disconnections can be caused intentionally or unintentionally by the patient, as well as by external circumstances, such as movements of equipment or devices in/at the bedside or on the patient.

Situations in which the patient changes position include, for example, situations in which the patient leaves a hospital bed, situations in which the patient moves in the hospital bed and situations in which the patient sits up in the hospital bed. Situations involving changes of location can be brought about by the patient intentionally or unintentionally.

For the purposes of the present invention, situational events during routine medical care and/or in the daily clinical routine are to be understood as any performance of care activities on the patient or with the patient.

For the purposes of the present invention, parameters of the cardiovascular system are to be understood as any indications or parameters that indicate a current health situation of the heart, circulation or hemodynamics, such as heart rate (HR), cardiac ejection fraction (EF), cardiac output (HMV), cardiac output (HZV), heart rate variability (HRV), stroke volume (SV), pulse rate or pulse rhythm, pulse quality, pulse wave transit times, systolic blood pressure (SYS-BP), diastolic blood pressure (DIA-BP), partial pressure of oxygen (pO₂), partial pressure of carbon dioxide (pCO₂) in the blood, carbon dioxide saturation (SPCO₂) or oxygen saturation (SpO₂) in the blood, subcutaneously or transcutaneously recorded saturations of carbon dioxide or oxygen (etCO₂, ptcCO₂), body temperatures, such as core temperature, proximal temperature, shell temperature, surface temperatures, skin temperatures.

For the purposes of the present invention, parameters of the vascular or lymphatic system or parameters of the blood circulation system are to be understood as any data indicating a state or composition of body fluids such as blood, urine, sweat, saliva, fluids of the brain, stomach, gall bladder, pancreas or lymph of the patient. Parameters of the blood circulation system also include parameters that can be determined by means of a blood gas analysis or blood analysis.

For the purposes of the present invention, measured variables (measurands) or parameters of the upper airways, the lower airways or the lungs are to be understood as any measured variables or parameters of pressure, flow rates, volume, respiratory rate, time courses of pressures, volumes, flow rates, breath, which characterize conditions in the upper or lower airways or in the lungs.

In the context of the present invention, variables or parameters derived from measured variables are understood to mean, for example, the work of breathing (WOB) performed by the patient.

In the context of the present invention, respiratory physiological parameters or ventilation parameters are any parameters of pressure, flow rate, volume, respiratory rate, time courses of pressures, volumes, flow rates, respiratory rate, which characterize or indicate settings, situations, events or measured variables during mechanical ventilation of a patient, as well as settings during mechanical ventilation of a patient which are associated with conditions in the upper or lower airways or in the lungs as well as clinical picture, diagnosis or therapy. These include in particular: tidal volume (VT), minute volume (MV), minute ventilation volume (AMV), breath volume, airway pressures, inspiratory ventilation pressure (Pinsp), expiratory ventilation pressure (Pexp), inspiratory flow rate (V′insp), expiratory flow rate (V′exp), inspiratory volume (Vinsp), expiratory volume (Vexp), respiratory rate or ventilation rate (RR), spontaneous minute volume (MV_Spon), spontaneous respiratory rate (RR_Spon), inspiratory-to-expiratory ratio (I:E ratio), gas concentrations (CO₂, etCO₂) in exhaled gas.

For the purposes of the present invention, an instruction is to be understood as any contact with the patient, addressing the patient in the form of a speech address to the patient by the support system. The instruction thus represents an address, which is addressed to the patient by the support system. The address or message through the instruction can be directed to perceptions of the patient such as warmth, cold or pain.

The address or message transmitted to the patient by the instruction can also be directed to at least one or all of the patient's senses, i.e. to the sense of hearing (auditory sensory perceptions), the sense of sight (visual sensory perceptions), the sense of taste (gustatory sensory perceptions), the sense of smell (olfactory sensory perceptions), the sense of touch or feel (tactile sensory perceptions). The instruction can be configured with one or more elements as well as combinations of several elements, which are suitable for addressing (messaging) at least one or various senses of the patient. These include, for example, visual or auditory instructions as audio, video, in the form of texts, speech, sound, music, text modules, sentences, sentence modules, sentence fragments, speech sequences, words, syllables, audio sequences, speech instructions, text representations, graphic representations, visualizations, film or video sequences, diagrams, symbols, pictograms, individual images, individual image sequences, for example in the form of animations (comics), animation sequences (comic sequences). For this purpose, suitable devices for audio playback and/or video display can be connected to the support system. Suitable pictograms are formed by images or symbols which can convey one or more pieces of information by means of simplified graphic representations. Instructions with comparable objectives or the same objective, which are physiologically or thematically related, can be structured as an instruction family and stored in the data memory. For example, instructions that are aimed at a similar specific behavior of the patient can form an instruction family. Such instructions are thus based on a common intention or purpose. Instructions within an instruction family can implement the same or comparable intentions through a different character. For example, the character of a voice instruction can be polite, friendly, even affectionate, but it can also be very definite or demanding. It is also possible to combine the character of the instruction with the intention and possible further properties in order to compile a kind of instruction set, which can then be used to give a specific instruction to an automatic speech synthesis system, given the intention, character and possible further property, which then generates the text for the next instruction to the patient—usually and exemplarily using artificial intelligence or neural networks.

An example “command” to a speech synthesis system and a text instruction as a fictitious possible “result”:

Command: “Formulate a friendly but firm instruction to breathe in (inhale) more slowly, in Swiss dialect” (a dialect of the German language); result: “Streng di mol mehr an, bewusst tief zue atme!” (translation: Make more effort to breathe deeply!).

In addition to the described possibilities of reaching the patient's senses through speech or images, addressing or messaging with the help of scents to stimulate the sense of smell and/or taste is also possible. Suitable devices for dosing, vaporizing or nebulizing volatile substances must be connected to the support system for this purpose.

In the context of the present invention, a confirmation address (feedback address) means any instruction or information that is provided to the support system and indicates or represents a reaction to an instruction previously issued by the support system to the same patient. This means that the patient changes or adapts his/her behavior in his current situation—for example under medical therapy, in care situations, in his/her environment, in the regular routine of the clinical daily routine—on the basis of the instruction or a content of the instruction. Confirmation addresses include, for example, reactions to instructions that contain instructions to change behavior. Confirmation addresses in the sense of the present invention can be based on the same sources of data or information as the data elements or as the data elements and vice versa. In this respect, it is included in the sense of the present invention that aspects which are described and explained for types, classes, groups of data elements and aspects which are described and explained for recording, determining, obtaining or calculating data elements are in principle also transferable to confirmation addresses and vice versa, so that in the context of the present invention mutual reference can be made in each case. Furthermore, the following claims are hereby incorporated into the detailed description, where each claim may stand as a separate example by itself. While each claim may stand alone as a separate example, it should be noted that although a dependent claim may refer to a particular combination with one or more other claims in the claims, other examples may also include a combination of the dependent claim with the subject matter of any other dependent or independent claim.

Such combinations are explicitly suggested herein, unless it is stated that a particular combination is not intended. Furthermore, features of a claim are also intended to be included for any other independent claim, even if that claim is not made directly dependent on the independent claim.

The support system according to the invention is suitable for supporting or assisting a patient during medical diagnostics or therapy.

Such a support system according to the invention has at least one interface, a control unit with an associated data analysis unit, a data storage unit for storing instructions, instruction families or text sequences of instructions and an output unit. The data analysis unit is configured to analyze the data elements and to provide an analysis result. The control unit is configured to use the analysis result to select at least one instruction from a group of instructions stored in the data storage unit and to provide the at least one selected instruction to the output unit.

The output unit is configured to provide the at least one selected instruction to the patient. The at least one interface is configured to capture at least one confirmation address.

The at least one confirmation address indicates a reaction or feedback from the patient to at least one instruction previously addressed and issued to this patient. The instructions stored in the data storage unit are configured to address or message at least one of the human senses such as hearing, sight, taste, smell, touch or feel. The interface arrangement (one or more interfaces) or at least one interface is configured to receive at least one data element from a group of data elements and to capture at least one confirmation address from a group of confirmation addresses.

The control unit is configured to coordinate the interface arrangement, the output unit of the data storage unit and the data analysis unit in the support system.

The control unit is configured to use the analysis result to select at least one instruction from a group of instructions stored in the data storage unit and to provide the at least one selected instruction to the output unit.

The data analysis unit is configured to analyze the data elements and to provide an analysis result. The output unit is configured to provide the patient with at least one selected instruction.

The data elements can be based on measured variables or variables derived from measured variables, on parameters, information, planning data, calendar data, time data, data and information provided to the support system by medical devices, databases, monitoring systems (Acute Care System), access control systems, camera systems, data processing systems or other data sources, which are provided to the support system in direct wired or wireless connection as well as in indirect wired or wireless connection via networks (LAN, WLAN), server system or a cloud system.

According to the invention, the control unit is configured to continuously select or adapt the instructions on the basis of the data elements and/or on the basis of the confirmation address during operation of the support system and to coordinate the provision to the output unit.

The one or more confirmation address indicates a response or feedback from the patient to at least one instruction previously addressed to and issued to that patient. At least one confirmation address comprises mental, physical, physical, neurological, physiological or metrologically determined data or information.

The confirmation address can be based on measured variables or variables derived from measured variables, on parameters, information, planning data, calendar data, time data, data and information provided by medical devices, databases, monitoring systems (Acute Care System), access control systems, camera systems, data processing systems or other data sources, which are provided to the support system in direct wired or wireless connection as well as in indirect wired or wireless connection via networks (LAN, WLAN), server system or a cloud system.

Embodiments present possibilities of addressing (messaging) the patient by the support system. In the context of the embodiments, reference is also made to the definitions, explanations and some examples given, in particular with regard to instructions, intentions, data elements, data sources and confirmation address at the beginning of the application text.

In embodiments, the group of data elements may comprise at least one of the following data elements:
data elements, which indicate physical, pathological, physiological or psychological situations of the patient,
data elements which indicate a parameter of the upper airways (upper respiratory tract), the lower airways (lower respiratory tract) or the lungs, a respiratory physiological parameter or a ventilation parameter, such as pressure levels, respiratory or ventilation frequencies, flow rates, volumes, compositions of inhaled or exhaled gases, wherein the data elements indicate an exceedance of the upper airway, lower airway or lung parameter, the respiratory physiological parameter or ventilation parameter above a predetermined threshold value for a predetermined period of time or for a predetermined number of times or wherein the data elements indicate a shortfall of the parameter of the upper airways, the lower airways or the lungs, the respiratory physiological parameter or the ventilation parameter below a predetermined threshold value for a predetermined period of time or for a predetermined number of times,
data elements which indicate a neurological parameter, wherein the data elements indicate an exceeding of the neurological parameter above a predetermined threshold value (LimitHigh) for a predetermined period of time or for a predetermined number of times, or wherein the data elements indicate a falling below of the neurological parameter below a predetermined threshold value (LimitLow) for a predetermined period of time or for a predetermined number of times,
data elements which indicate a physical parameter, wherein the data elements indicate an exceedance of the physical parameter above a predetermined threshold value (LimitHigh) for a predetermined period of time or for a predetermined number of times or wherein the data elements indicate a falling below of the physical parameter below a predetermined threshold value (LimitLow) for a predetermined period of time or for a predetermined number of times,
data elements which indicate a parameter of the cardiovascular system or of the blood circulation, vascular or lymphatic system, such as pressure levels or flow or volume situations, wherein the data elements indicate an exceedance of the parameter of the cardiovascular system or of the blood circulation, vascular or lymphatic system above a predetermined threshold value (LimitHigh) for a predetermined period of time or for a predetermined number of times or wherein the data elements indicate that the parameter of the cardiovascular system falls below a predetermined threshold value (LimitLow) for a predetermined period of time or for a predetermined number of times,
data elements that characterize physiologically or psychologically induced situations of pain, anxiety or panic, such as
   by an increase in the heart rate (HR) within a predetermined time interval above a predetermined threshold value (LimitHigh),
   increase in skin moisture within a predetermined time interval above a predetermined threshold value,
   increase in respiratory rate (RR) within a predetermined time interval above a predetermined threshold value (LimitHigh),
   change in muscle tone,
   increase in systolic blood pressure (SYS-BP), diastolic blood pressure (DIA-BP),
data elements that indicate alarm situations during medical therapies, diagnosis, care measures or during the regular clinical daily routine,
data elements that indicate situations with activities of the patient in the patient's environment or movement events in connection with the patient in a therapy or treatment environment or in care situations,
data elements that indicate situations or events in a therapy or treatment environment of the patient during the performance of medical therapies, diagnoses, care measures or the regular routine in the clinical daily routine,
data elements that are based on two-dimensional or three-dimensional image or video data and indicate the patient's condition or events in relation to the patient's environment,
data elements that are based on two-dimensional or three-dimensional image or video data and indicate situations of potential changes in the patient's location,
data elements that are based on two-dimensional or three-dimensional image or video data and indicate situations of potential disconnections of the patient from their connection lines or access lines,
data elements that indicate status data or alarms of other patients or events,
data elements that indicate status data or alarm situations of the patient or events in relation to the patient, whereby the data elements are based on audio data, such as alarm signals from the direct therapy or treatment environment or a spatially close environment of the patient.

In the context of the present application, some data elements from the above listing of the group of data elements are explained in relation to the clinical application by means of examples—also with reference to figures and tables—in the context of configuration possibilities of instructions from the above listing of the group of instructions with regard to possible subsequent address (message) to the patient in the clinical setting—also with reference to figures and tables. Some of the data elements of the group of data elements listed in the context of this preferred embodiment are briefly explained here in more detail with regard to the context and the indexing of situations related to the patient and with regard to the use by the support system and the advantages thereby brought about by means of the support system.

Exemplary data elements that indicate patient movement events can be provided to the support system, for example, on the basis of two-dimensional or three-dimensional image or video data obtained from an imaging or camera system or an acute care system. The following patient activities, for example, can be assigned to such movement events: sitting up in the hospital bed, adjustments in the lying position of the hospital bed, repositioning in the hospital bed, leaving the hospital bed, walking through the room, taking drinks or meals, reading, watching television, cleaning the body and personal hygiene. Alternatively, leaving the hospital bed can also be indicated by a scale in the lying surface of the hospital bed, provided the hospital bed is equipped accordingly.

Exemplary data elements that indicate situations in a therapy or treatment environment are, for example, regular non-invasive blood pressure measurements, during which a blood pressure cuff is inflated on the patient's upper arm. Other data elements that indicate situations or events in a therapy or treatment environment are, for example, regular actions in the daily routine such as visits, administration of medication, care measures, washing or the consumption of meals.

Some of the data elements—in particular measured values, physiological or respiratory physiological parameters—can be evaluated in relation to falling below a predetermined lower threshold value (LimitLow) or exceeding a predetermined upper threshold value (LimitHigh)—a threshold violation. In this way, a deviation from normal ranges can be identified and evaluated as an indication that the patient is experiencing a special situation or constellation (scenario). In the vast majority of such special situations or scenarios (constellations), the situation is such that it can be assumed that such situations or scenarios (constellations) are associated with stress for the patient. In combination with an evaluation of two-dimensional or three-dimensional image or video data or audio data, the identified special scenario (constellation) or situation can be checked for plausibility. Then, in order to reduce the patient's stress level, a calming address (message) to the patient can be the procedure of choice in such cases. Preferably, a reference to the event causing the threshold violation in context is made in the address (message)—for example by means of voice output. An example of this can be given in connection with the measurement of oxygen saturation (SpO₂) in the blood, if a sensor alarm was caused by excessive movement of the SpO₂ finger clip sensor: “Stay still, you are moving your arms or hands too much, so there are incorrect measurements on the sensor attached to your finger: Keep your arms and hands a little more steady!” Another example of this can be given in connection with an identified increase in respiratory rate (RR), i.e. if the patient breathes in too hectically or too quickly: “Stay calm, breathe in more calmly and slowly . . . and out . . . in . . . and out again . . . , and calm down!” When giving such instructions to calm down, meditative aspects can also be incorporated into the instructions.

In preferred embodiments, the instruction stored in the data storage unit in a group of instructions as at least one instruction contains or has: intentions, text modules, sentence modules, sentence fragments, speech sequences, words, syllables, audio sequences, speech cues, sound reproductions, music reproductions, sound reproductions, sound reproductions, text representations, graphic representations, visualizations, film or video sequences, individual images, individual image sequences, diagrams, symbols, pictograms. Some of the instructions of the group of instructions listed in the context of this preferred embodiment are briefly explained here in more detail with regard to the context and the indexing of situations related to the patient and with regard to the use by the support system and the advantages conferred by the support system.

In preferred embodiments, the group of confirmation addresses may comprise at least one of the following confirmation address:
Confirmation address that indicate physical, pathological, physiological or psychological situations of the patient,
Confirmation address that indicate an upper airway, lower airway or lung parameter, a respiratory physiology parameter or a ventilation parameter compared to a time before the instruction was issued to the patient,
Confirmation address that contain a parameter of the lungs, of the cardiovascular system or a parameter of the circulatory, vascular or lymphatic system, such as heart rate such as heart rate (HR), heart rate variability (HRV), systolic blood pressure (SYS-BP), diastolic blood pressure (DIA-BP), wherein the confirmation address indicate a change in the parameter of the cardiovascular system or the parameter of the blood circulation or vascular system compared to a point in time before the instruction was issued to the patient,
Confirmation address that indicate physiologically or psychologically induced situations by a change in heart rate (HR), skin hydration, respiratory rate (RR) or muscle tone compared to a time before the instruction was given to the patient,
Confirmation address that indicate events or situations, status data during medical therapies, diagnoses, care measures or during the regular clinical daily routine,
Confirmation address that indicate situations with movement events in a therapy or treatment environment, in the care situation or in the patient's environment,
Confirmation address that indicate situations with activities in the patient's environment, movement events in connection with the patient in the therapy or treatment environment or in care situations,
Confirmation address that are based on two-dimensional or three-dimensional image or video data and indicate situations with regard to the status of the patient's connection lines or access lines compared to a point in time before the instruction was issued to the patient,
Confirmation address that indicate situations based on two-dimensional or three-dimensional image or video data of potential changes in the patient's location relative to a point in time before the instruction was issued to the patient,
Confirmation address that indicate changes in alarm situations compared to a point in time before the instruction was issued to the patient.

Some of the confirmation address of the group of confirmation addresses listed in the context of this preferred embodiment are further explained herein in relation to the context and indication of situations related to the patient and in relation to the use by the support system and the benefits brought about by the support system. In addition, in the context of the present application, some embodiments of confirmation address from the above listing of the group of confirmation addresses are explained with reference to the clinical application by means of examples—also with reference to figures and tables.

Further preferred embodiments show that the control unit can be configured to assign a prioritization to the instructions and to configure the temporal sequence of the output of the instructions to the patient on the basis of the prioritization. The control unit can prioritize the instructions when providing and/or issuing the instructions. An example of prioritization is a situation in which the support system has identified two situation features (characteristics) based on measured values (HR, RR) and on two-dimensional or three-dimensional image or video data:
Situation feature 1: The patient is in a stressful situation, breathing rapidly, for example with a breathing rate of more than 20 breaths per minute, and has an increased heart rate, for example a heart rate of 110 beats per minute.
Situation feature 2: The patient acts on or with the infusion line.

Based on situation feature 1, the patient is instructed to breathe in calmly and consciously, while situation feature 2 instructs the patient to stop interacting with the infusion line. In contrast to situation feature 1, situation feature 2 indicates an imminent threat to the patient if the patient were to manage to pull out the infusion line, which is why the instruction to the patient to stop interacting with the infusion line is given a higher priority and issued to the patient immediately, while the instruction relating to breathing is put on hold for the time being. The prioritization of instructions therefore offers the advantage of being able to avert immediate danger to the patient.

Further preferred embodiments show how instructions can be classified or assigned to classes by the data analysis unit and/or the control unit with regard to the effect acting on the patient. Classification by the control unit with regard to the effect on the patient offers the advantage that subsequent instructions can be estimated or predicted with regard to possible subsequent effects. For example, if for an individual patient an instruction with a certain character reproducibly leads to better cooperation than instructions with a different character, then the control unit can select the same character for subsequent instructions to this patient and in this way adapt and take into account the addressing and messaging in a patient-specific manner.

Further preferred embodiments show how confirmation address can be based on two-dimensional or three-dimensional image or video data or on audio data. A mood of the patient can be determined from two-dimensional or three-dimensional image or video data or audio data by the data analysis unit by evaluating a facial expression, a facial expression or a physiognomy from the two-dimensional or three-dimensional image or video data by the control unit. In this way, for example, the mood state—approval, rejection, joy, sadness, pain, relief, horror, fear and panic—can be read from the face. This allows the control unit to evaluate whether the patient is cooperating or whether an instruction given to the patient has been received positively or negatively by the patient. Furthermore, the patient's state of mind or mood (or mood state) can be determined on the basis of audio data, whereby the audio data can be evaluated by the control unit to determine the patient's state of mind or mood with regard to the sounds made by the patient, which are contained in the audio data in the form of spoken words or sentences, crying, laughter or the patient's cry of fear, for example. In this way, for example, approval, rejection, joy, sadness, pain, relief, horror, fear and panic can be analyzed in the audio data. The control unit can also combine the image or video data with the audio data in order to derive, for example, situations of approval, rejection, joy, sadness, pain, relief, horror, fear or panic of the patient. Recognition of the patient's mood or emotional state by the support system offers the advantage that, in addition to or as an alternative to data or parameters recorded by measurement technology, it is possible for the support system to record the patient's reactions or the effects on the patient of instructions previously addressed to the patient directly—as if they could be read from the patient's face—and then to configure further support based on this, for example by repeating or adapting further addressing (speeches/messaging).

In a further preferred embodiment, the data analysis unit can determine a spatial situation of the patient from the two-dimensional or three-dimensional image or video data. Possible changes in the patient's location in the treatment environment can be determined from a current room situation in comparison with previously recorded room situations. Changes in the patient's position in the treatment environment, in the care situation or in the therapy environment—for example in the patient's room—can be, for example, sitting up in the patient's bed, leaving the patient's bed or movement situations of the patient such as lying down, walking or sitting in the patient's room.

Embodiments show how at least one data element and/or at least one feedback element can be provided from different data sources with the use of interfaces. At least one possible data source can be mentioned here as an example: a medical device, a diagnostic device, a therapy device, in particular a ventilator or an anesthesia device, a medical measuring device, a physiological patient monitor (PPM), a blood gas analysis device, an electroimpedance tomography device (EIT), an ultrasound imaging device (US), an X-ray device (X-Ray), a mammography device, a heart sound contraction recorder (CTG), a computer tomography device (CT), a magnetic resonance imaging (MRI) device, an imaging or camera system, a thermometer for recording body temperatures, a device for determining the carbon dioxide saturation or oxygen saturation in the blood or a device for determining the carbon dioxide concentration in respiratory gases, such as a capnometer or oxy-capnometer, a heat therapy device, such as a warming bed or incubator. The advantage of connecting the aforementioned data sources to the support system is that it enables the control unit of the support system to compare data relating to the patient's situation. For example, data from the physiological patient monitor (PPM) can be compared with data from the ventilator in order to determine whether an increase in heart rate is accompanied by an increase in respiratory rate. This enables the control unit to analyze the patient's current situation with improved reliability and to evaluate the patient's possible reactions to addressing and instructions with improved reliability. In this example, if the address (message) to the patient “Please breathe more calmly” had been successful, parameters of the ventilator (RR, VT, MV) and parameters of the physiological patient monitor (HR, BP) would have been used to evaluate the effect of the address (message). A reduction in heart rate with an almost constant respiratory rate and low tidal volume are a possible indication that the patient is calmed as a result of the address (message), but also indicates comparatively shallow breathing. A reduction in heart rate and respiratory rate indicates comparatively deeper and conscious breathing, as a result of which the heart rate is significantly reduced and can be considered an indication that the patient is calming down as a result of the address (message).

Embodiments show how the support system can be used as a part or as a component of a medical device, a diagnostic device, a therapy device, in particular a ventilator device or an anesthesia device, a medical measuring device, a physiological patient monitor (PPM), an electroimpedance tomography device, an ultrasound imaging device, an X-ray device (X-Ray), a mammography device, a heart sound contraction recorder (CTG), a computer tomography (CT) device, a magnetic resonance imaging (MRI) device, an imaging or camera system, a thermometer for detecting body temperatures, a device for measuring carbon dioxide saturation or oxygen saturation in the blood, a device for determining the carbon dioxide concentration in respiratory gases, such as a capnometer or oxy-capnometer, a heat therapy device, such as a warming bed or incubator. In embodiments in which the support system is configured as a part or as a component of one of the devices of the aforementioned devices, there are advantages in data exchange, in particular with regard to real-time capability in data exchange. For example, in situations of singular events, such as a very rapid increase in heart rate due to a shock, it can prove advantageous if the support system is configured as an integral part of the physiological patient monitor (PPM) and is thus connected to the real-time data communication of the physiological patient monitor (PPM). For example, in situations of singular alarm events, such as a low tidal volume (VT_Low) alarm, it can prove advantageous if the support system is configured as an integral part of the ventilator and is thus connected to the real-time data communication of the ventilator, in order to compare the alarm with two-dimensional or three-dimensional image or video data of the patient at the same time, for example, in order to recognize whether there is or was a blockage in the connecting lines of the respiratory gas supply, which could have been the possible cause of the alarm.

In a particularly preferred embodiment, the control unit can be configured to continuously select, generate, filter or adapt the instructions on the basis of the data elements and/or on the basis of the confirmation address during operation of the support system. The control unit can be configured to include at least one patient-specific criterion from a group of patient-specific criteria, such as the following, in the selection, generation, filtering or adaptation of the instructions:
name, nationality,
mother tongue, gender, sexual orientation,
language, mother tongue, dialect, sign language,
age, height, weight, body mass index (BMI),
mood, state of mind,
clinical picture, diagnosis, medication, anesthesia,
therapy situation, care situation, degree of therapy, degree of care, duration of therapy,
physical, pathological, physiological or psychological situations of the patient,
type and balance of diet (fats, carbohydrates, carbohydrates),
dietary arrangements, in particular special diets,
information from a nutrition and fluid management system,
length of stay in the therapy or treatment environment,
privacy in the therapy or treatment environment.

In certain situations during the course of support, it may be useful to adapt the instructions in terms of intention or character. For example, for some patients it may be necessary to address any further address with a very strong tone and emphasis if previous attempts to address (message) the patient in a friendly manner have failed to motivate them to participate or cooperate.

In certain situations during the course of support, it may be useful to filter the instructions by suppressing one or more address. If, for example, care staff are talking to the patient or performing actions on them at the same time, this can lead to unwanted irritation. Filtering certain instructions is also appropriate if the patient has repeatedly failed to respond (react) to identical intoned instructions. Regardless of the cause, this can prevent further disturbances to the patient. One criterion for instruction filtering can be, for example, the number of repetitions (address frequency) of instructions.

If a certain number of instructions per time unit (e.g. per hour, per day) is exceeded, it can be assumed that the instructions may be perceived as disturbing by the patient. The general state of mind (mood, emotional state), alertness or the patient's medication can be included in the instruction filtering as additional information.

In certain situations during the course of support, it may be beneficial to adapt the instructions to specific situations, for example to address a patient in their native language or a dialect in order to increase the chance of the patient's willingness to participate. Different, target group-oriented approaches depending on nationality, religion, gender or age can also help to improve the patient's willingness to participate and contribute to the therapy.

Embodiments or combinations of embodiments can show which technical configurations the support system can use to establish contact, i.e. to address (message) the patient. Acoustic addressing with voice announcements, sounds, music, tones or noises can take place by means of headphone systems, structure-borne sound hearing systems or loudspeaker systems in wireless or wired form. Wireless technologies such as Bluetooth, WLAN or DECT can be used. Visual output can be implemented using screens or ceiling monitors mounted above the patient as well as data glasses (virtual reality/augmented reality), for example, so that the content displayed can be perceived regardless of the patient's position in the care environment or in bed.

An embodiment according to the invention is formed by a process for supporting a patient during the performance of medical treatments, therapies, diagnoses.

A further embodiment according to the invention is formed by a process for operating a support system for supporting a patient during a performance of medical treatments, therapies, diagnoses according to one of the previous embodiments.

A process for supporting a patient and the process for operating a support system is divided into performing a continuously repeating sequence of the following steps:
a) reading in at least one data element from a group of data elements;
b) evaluating of data elements with regard to situations or events that are related to a patient's condition or behavior;
c) selecting an instruction to the patient from a group of instructions or instruction families;
d) issuing the instruction to the patient;
e) reading in at least one confirmation address from a group of confirmation addresses;
f) evaluating the confirmation address with regard to situations or events related to a patient's condition or behavior.

This sequence of steps can be coordinated by a control unit, for example.

A further embodiment according to the invention is formed by a computer program on or with non-transitory computer-readable medium (e.g., fixed in tangible media) or computer program product on or with non-transitory computer-readable medium (e.g., fixed in tangible media) comprising a program code for carrying out the process for assisting a patient or a process for operating a support system. The program code may preferably be executed on a computer, a processor or a programmable hardware component. Steps, operations or processes of various processes described above may be performed by programmed computers or processors.

Examples may also cover program storage devices (non-transitory computer-readable medium), such as digital data storage media, that are machine-, processor-, or computer-readable and encode machine-executable, processor-executable, or computer-executable programs of instructions. The instructions perform or cause to be performed some or all of the steps of the processes described above. The program storage devices may include or be, for example, digital storage devices, magnetic storage media such as magnetic disks and magnetic tapes, hard disk drives, or optically readable digital data storage media.

Further examples may also cover computers, processors or control units programmed to perform the steps of the processes described above, or (field) programmable logic arrays ((F)PLAs) or (field) programmable gate arrays ((F)PGAs) programmed to perform the steps of the processes described above.

Only the principles of the disclosure are illustrated by the description and drawings. Further, all examples herein are expressly provided for illustrative purposes only to assist the reader in understanding the principles of the disclosure and the concepts contributed by the inventor(s) to the advancement of the art. All statements herein concerning principles, aspects and examples of the disclosure, as well as specific examples thereof, include equivalents thereof. It is to be understood that the disclosure of several steps, processes, operations or functions disclosed in the description or claims is not to be construed as being in the particular order unless explicitly or implicitly stated otherwise, e.g. for technical reasons. Therefore, the disclosure of multiple steps or functions does not limit them to a particular order unless these steps or functions are not interchangeable for technical reasons. Further, in some examples, a single step, function, process or operation may include and/or be broken into multiple sub-steps, sub-functions, sub-processes or sub-operations. Such sub-steps may be included and form part of the disclosure of that single step, unless they are explicitly excluded.

Below are some examples of the advantages and possibilities resulting from addressing the patient with instructions using confirmation (feedback) address.

In some situations, it may be necessary to reinforce certain requests to the patient—for example by repeating the address. If, for example, situations involving disconnection of the patient's access lines for the supply or continuation of fluids or gas volumes, for example in the form of self-extubation with removal of the endotracheal tube, breathing mask or tracheostoma or in the form of self-decannulation with removal of catheters or invasive cannulas by the patient, can be identified at an early stage, an attempt can first be made to dissuade the patient by addressing (messaging) them with friendly and reassuring words. In the next step, the request can be further emphasized by addressing (messaging) the patient clearly. The confirmation address can therefore be used to determine whether and to what extent the patient reacts to the instructions and what degree of cooperation or willingness to participate the patient shows in the current situation, in order to then adapt the instructions in the manner or style as explained. The way in which instructions are given to the patient can influence their effect, even if these instructions are equivalent in content. For example, the patient's state of mind (mood, emotional state), the time (morning, noon, evening, night), their alertness and receptiveness can play a significant role in the patient's acceptance of the voice instructions. If the instructions are equivalent in content, the use of the respective text sequences or phrases, as well as the avoidance of repetitions of identical text sequences or phrases, can also have an influence on the effect and/or acceptance of the instructions.

In the following, Table 1 provides an overview of an exemplary configuration of the support system in combination with a ventilator and lists the necessary conditions for some events during mechanical ventilation. Situations with alert events are listed, as well as situations with impending alert events that have been identified by the ventilator on the basis of the content of data elements and which the patient may be able to avert by taking appropriate action. The impending alarm events can also be referred to as pre-alert events.

The data elements according to this Table 1 indicate, for example, in particular ventilation settings, ventilation parameters, measured values from sensors, in particular pressure sensors and/or flow rate sensors, as well as measured variables derived from the sensors, such as inspiratory and expiratory volumes, and variables derived from inspiratory or expiratory flow rate measured values by means of integration over defined time intervals.

TABLE 1

| | | Alert Event/Pre- | Instruction/ |
|---|---|---|---|
| | Content of Data Element | Alert Event | Address |
| a.) | VT_{measured value} < VT_{LimitLow} + | PreVTLow: Tidal | AlertMess_1: |
| | 20% for 3 breaths | volume decreases, | Encourage patients to |
| | | VTlow alarm | breathe more deeply |
| | | imminent | |
| b.) | VT_{measured value} > VT_{LimitHigh} − | PreVTHigh: Tidal | AlertMess_2: |
| | 20% > for 3 breaths | volume increases, | Persuade patients to |
| | | VThigh alarm | breathe more |
| | | imminent | shallowly |
| c.) | MV_{measured value} < MV_{LimitLow} + | PreMVLowByVT: | AlertMess_3: |
| | 20% plus indicator for too low VT | Tidal volume | Encourage patients to |
| | | decreases, MVlow | breathe more deeply |
| | | imminent | |
| d.) | MV_{measured value} < MV_{LimitLow} + | PreMVLowByRR: | AlertMess_4: |
| | 20% plus indicator for RR too low | Respiratory rate | Encourage patients to |
| | | decreases, MVlow | breathe more |
| | | imminent | frequently |
| e.) | MV_{measured value} > MV_{LimitHigh} − | PreMVHighByVT: | AlertMess_5: |
| | 20% plus indicator for too high VT | Tidal volume | Persuade patients to |
| | | increases, MVhigh | breathe more |
| | | imminent | shallowly |
| f.) | MV_{measured value} > MV_{LimitHigh} − | PreMVHighByRR: | AlertMess_6: |
| | 20% plus indicator for RR too high | Respiratory rate | Persuade patients to |
| | | increases, MVhigh | breathe more slowly |
| | | imminent | |
| g.) | RR_{measured value} > RR_{LimitHigh} − 20% | PreRRHigh: | AlertMess_7: |
| | | Respiratory rate | Persuade patients to |
| | | increases, RRHigh | breathe more slowly |
| | | imminent | |
| h.) | Paw_{measured value} > Paw_{Coughing} plus | Coughing: Cough | AlertMess_8: |
| | indicator for coughing | | Reassure patients |
| i.) | Paw_{MeasuredValue} > Paw_{CounterBreathing} | CounterBreathing: | AlertMess_9: |
| | plus indicator for counter breathing | Counter-breathing | Encourage patients to |
| | | of the patient | breathe |
| | | | synchronously with |
| | | | the device, |
| | | | optional: Support |
| | | | through the output of |
| | | | an acoustic |
| | | | respiratory rhythm |
| | | | signal |

Here is an example of the situation g.) (PreRRHigh), selected as an example from Table 1, in which a patient is breathing at an increased respiratory rate. The pre-alarm event is fulfilled if a measurement of the respiratory rate (RR) results in the following situation:

$$RRMeasured value > RRLimitHigh - 20 \% ,$$

i.e. the measured breathing rate is greater than an associated upper alarm limit minus a defined offset of 20%, for example. The offset ensures that the condition for issuing the instruction is fulfilled before the actual alarm is issued. The size of the selected offset can be configured to depend in particular on the patient's reaction time to acoustic instructions. Advantageously, the offset should therefore be adaptable both to the individual patient and over the course of therapy. In practice, an increase in the breathing rate could, for example, be due to a high situational stress level of the patient. By explaining the current situation and providing instructions for calmer breathing, the patient can be supported in actively reducing the respiratory rate and thus counteracting a “high respiratory rate” alarm in good time.

Embodiments can show how the support system can be configured as an adaptive support system with adjustments to changes using trend analyses as an example. Trend analyses can also be used to detect gradual changes in patient parameters. For example, the patient can be guided by specific instructions through a predetermined “therapy corridor”, which is defined by the corresponding measured values—as listed in Table 1 for exemplary embodiments of a support system in combination with a ventilator—and their alarm limits.

Embodiments can show how the support system can be configured as a support system with instruction selection by filtering data elements, for example. The control unit preferably uses rules to filter the data elements. An essential rule here is that no contradictory instructions are transmitted to the patient on the basis of different data elements after filtering has taken place.

For example, a patient is breathing very irregularly, i.e. deep and shallow breaths follow each other, so that alarm situations with a tidal volume that is too high (VT_High) and alarm situations with a tidal volume that is too low (VT_Low) could be imminent. An announcement that the patient should breathe more shallowly but at the same time more deeply would not make sense. In such a case, an attempt could be made to reassure the patient if stress is possibly the cause of the irregular breathing and an announcement could be made to the patient to breathe more consciously and evenly.

Embodiments can show how the support system can be configured as a support system with instruction prioritization by means of a prioritization of data elements. In preferred embodiments, instructions can be categorized in so-called “instruction families”. An instruction family describes the semantics of the address, but not the specific expression. One and the same instruction family always contains instructions with the same content, which can be both verbal and non-verbal (e.g. sounds or noises). It is essential that all instructions in a language family have the same effect on the patient. Each specific instruction text should be formulated as vividly and colloquially as possible in order to create a high level of acceptance among patients and avoid the impression of a “stupid machine announcement” as far as possible. In order to take into account the patient's general state of mind (mood, emotional state), the patient can be encouraged or reassured by varying the pitch of the voice instructions. It is generally advisable to change the pitch of the voice from time to time in order to reduce habituation effects. In addition to a change in the way the voice is used, instructions can be reinforced by giving them with a special intonation and/or at a higher volume. For this purpose, corresponding files (sound files) with different voice pitches (natural voice, neutral voice, automatic voice, male voice, female voice, sexy voice, soft voice pitch, loud voice pitch, pronunciation with special emphasis) can be stored in the data storage unit and kept ready by the control unit for customized provision to the output unit.

Processes for the synthetic generation of text instructions or text sequences as well as processes for speech and/or sound synthesis can also be used by the control unit or the output unit to create different voice levels.

The following embodiment serves as an example to holistically illustrate an implementation of the support system with options for event identification, event-to-instruction assignment, instruction selection, filtering of events and/or instructions, prioritization of events and/or instructions. Filtering and prioritization of events and/or instructions can preferably be carried out using properties, characteristics or criteria. Such properties, characteristics or criteria can, for example, be stored in the data storage unit in the form of sets of rules, assignment rules, formulas, reference or threshold values for the number and/or frequency of events or situations, for example in data tables, data fields (array, vector, matrix). The instructions, for example text instructions or voice instructions, can preferably be stored in image files (JPG, PNG, WMF) with symbols or pictograms or audio files (sound files, MP3, AAC) with audio sequences in the data storage unit in the form of libraries, data tables or as data records in a database.

In addition to the factual statements on the identified event, such instructions can also be assigned a character of the instruction, for example an informative character or a prompting character.

A statement of an informative nature is suitable, for example, to inform the patient about noises in their environment and to alleviate any uncertainty and any resulting fears or apprehensions.

A prompting statement is used to involve the patient in the therapy to encourage, praise or admonish them. For example, if the patient's breathing activity is too low, they can be encouraged to intensify their breathing activity, which can have the advantage of minimizing alarms during ventilation and increasing the patient's comfort.

The event identification and event assignment is carried out by the control unit using the data elements that are provided to the control unit via the interface. The identified events or situations are first mapped to instruction families. All specific instructions with identical information content are mapped under an address family.

Table 2 below shows how to motivate the patient to breathe more slowly using a family of instructions that includes different types of voice prompts with different characteristics.

TABLE 2

| ID/Sub-ID | Family ID = 7 | Score |
|---|---|---|
| 7.1 | “We analyze the breathing, Please breathe | 1 |
| | consciously.” | |
| 7.2 | “Please breathe in and out slowly.” | 2 |
| 7.3 | “You're breathing very quickly. Try to breathe | −1 |
| | a little slower.” | |
| 7.4 | “Good! Continue concentrating on your breathing - | 3 |
| | try to extend the pause a little longer after the next | |
| | exhale.” | |
| 7.5 | “You're breathing more calmly - very good! | 5 |
| | Try breathing a little slower.” | |
| 7.6 | “You're breathing too fast, that's not good! | −5 |
| | Try breathing slower.” | |

The instruction family used as an example in Table 2 (Family ID 7) has an identifier (Family ID) for an acoustic voice announcement of 7.

The character of the instruction ranges from informative, encouraging, prompting to confirming or praising. The confirmatory character can be selected if the confirmation address indicate that the patient's cooperation has been achieved due to previous encouraging or prompting speeches, i.e. the patient's willingness to participate has been identified and this willingness to participate can then be maintained and/or strengthened in the patient by means of praise. The character of the instructions can, for example, be scaled (ranked) using an identifier from “1”=“informative” to “5”=“high praise” or even to the negative identifier “−5”=“strong reprimand”.

As an example, the instructions are stored in the data store with the identifiers (ID, sub-ID).

The above case example with final and then praising address (message) to the patient shows the interaction of data elements indicating a ventilation parameter, their evaluation with subsequent selection of an instruction with an associated character, re-evaluation of further data elements that indicate ventilation parameters, renewed evaluation and selection of an instruction with an associated character as a feedback loop (closed loop).

Table 3 below shows some clinical events and situations that can be assigned to instructions with priority assignments on a scale of 1 to 5. To simplify the presentation, several comparable situations and/or events are summarized.

These events themselves are also broken down in more detail in Table 1.

TABLE 3

| Event | Instruction | Priority |
|---|---|---|
| (1) Therapy event: | Reassurance, explanation, | 1 |
| Recognition of intentions | instruction (omission) | |
| to act (standing up, self- | | |
| extubation, catheter | | |
| removal, . . .) | | |
| (2) Ventilation event: | Prompting the patient to adjust | 2 |
| “silent” pre-alarm | breathing, including motivation | |
| (indicator of an imminent | and reassurance (see Table 1) | |
| alarm without signaling) | | |
| (3) Ventilation event: | Reassurance | 2 |
| Cough (swallowing, | | |
| sputum) | | |
| (4) Ventilation event: | Explanation, reassurance; | 2 |
| counter-breathing | assistance by playing | |
| | (breathing) rhythms | |
| (5) Daily routine event: | Reassurance, orientation | 3 |
| Waking up, waking up with | instructions | |
| uncertainty (stress, | | |
| anxiety, pain) | | |
| (6) Anxiety situation: | Explanation, reassurance | 3 |
| Unfamiliar noises, actions | | |
| in the environment or on | | |
| the patient | | |
| (7) Pain situation: | Reassurance, announcement | 3 |
| Recognition via muscle | of help | |
| tension, stress, HR, HRV, | | |
| facial expressions) | | |
| (8) Unknown noises (technical | Explanation of the sounds | 3 |
| alarms or alarms from other | | |
| patients' devices, noises | | |
| from monitors, ventilators, | | |
| pumps, dishwashers, etc.) | | |
| (9) Stress event (noise) | Explanation, reassurance | 3 |
| (10) Time events (daily | Information, and possible | 4 |
| maintenance) | explanation of the | |
| | corresponding measure | |
| (11) Time events: | Greeting, orientation notes | 4 |
| e.g. wake-up calls | (place, time) | |
| (12) Therapy plan event: | Information, and possible | 4 |
| blood pressure measurement, | explanation of the | |
| recruitment, suction | corresponding measure | |
| (13) Environmental event | Timely announcement | 5 |
| e.g. visit, visit | | |
| (friends/family) | | |

In addition to the events/instructions, Table 3 also contains priority assignments. Here the priorities mean
1 immediate danger to the patient,
2 averting alarms,
3 alleviation of anxiety or stress in the patient,
4 preparations for upcoming care/therapy measures,
5 other information.

In a situation with priority 1, if the event or situation cannot be prevented by the patient's cooperation or otherwise, this results in a situation with a possible risk to the patient combined with a time commitment of clinical staff. In a situation with priority 2, if the event or situation cannot be prevented by the patient's cooperation or otherwise, this results in a situation involving effort and time commitment on the part of clinical staff. As a rule, individual events will occur with a time lag in everyday clinical practice. However, situations or combinations of situations can also arise. Situations may arise in which several situational events occur in quick succession, so that the time intervals between the resulting individual instructions or responses (reactions) would be so short that the patient may not be able to process or react to them rationally or mentally. In such a case, it is therefore advisable to both prioritize and filter the identified events and the resulting instructions before addressing the patient. The addressing can also be provided by the control unit to the output unit in a kind of queue according to the respective priorities.

The queue can contain instructions that have already been issued for a predetermined time interval, for example 30 minutes, as well as instructions that have not yet been issued. The filtering can be carried out, for example, in such a way that instructions with the same meaning or identical instructions as well as instructions of the same instruction family within the queue are included in the queue with a limit on the number and instructions that exceed the threshold on the number do not remain in the queue.

The instructions are preferably placed in a queue by the control unit and made available to the output unit. In addition to an instruction that is still waiting to be addressed to the patient, the queue can also include instructions that have already been issued over a predetermined elapsed time interval, for example 30 minutes. By means of filtering, the control unit can, for example, eliminate identical instructions or instructions belonging to the same instruction family from the queue within the queue. Filtering can also be used, for example, to correct or remove contradictory instructions in or from the queue. The control unit uses prioritization to ensure that instructions with the highest priority are placed in a position within the queue, which results in the immediate output of the corresponding address or instruction to the patient. Filtering offers the advantage of avoiding flooding the patient with instructions.

To select which of the instructions of the instruction family 7 is selected in this example, filtering and prioritization can be carried out methodically by means of a weighting, whereby the weight (credit) indicates, for example, which priority is assigned to the instruction and/or how often the instruction is present in the queue overall and how often an address with this instruction or with instructions from the same instruction family is still pending for output. The frequency and/or the weight (credit) can be used to adjust the prioritization within the queue. The respective weight (credit) is reduced, for example, after the instruction has been issued and then increased again to a maximum value or starting value after a predetermined time interval—preferably in several time-dependent steps.

Embodiments can show how the support system with an interface arrangement (one or more interfaces) for data exchange, data import and/or data export and/or for data input and/or output can be configured as an extended support system by way of example. The interface(s) can be integrated with additional functionalities for signal adaptations and/or protocol conversions as a unit in a network environment or in the medical device, for example in a ventilator. Data can be exchanged via SDC (IEEE 11073: Service-Oriented Device Connectivity), for example. In particular, this can enable status information to be fed back to the medical device or ventilator, for example to enable the operation of the medical device, in particular the operation of the ventilator, for example when performing ventilation, during measurement maneuvers and/or when initiating ventilation maneuvers. Data from other devices, medical devices, data sources such as physiological patient monitors (PPM), patient data management systems (PDMS), cameras, microphones, nurse call systems, patient context monitoring systems (Acute Care Monitoring, ACCM) can be included via the interface, provided they are also integrated via the network. These devices provide the data elements to the control unit via the interface. Events or situations are identified from the data elements, which then serve as the basis for generating, selecting or adapting instructions to the patient.

Embodiments can show how the support system can be trained to use speech synthesis processes to adapt, generate or compile the respective instructions during operation of the support system in a situation-specific manner. Processes of speech synthesis can, for example, make use of artificial intelligence processes in order to generate an instruction from sets of already existing collected formulated texts and documents.

Embodiments can show how the support system can be integrated into a user interaction with the clinical staff by means of user interfaces. Parallel to the acoustic output of instructions to the patient, an additional output option for the nursing staff is also conceivable in order to output the current patient status as well as the individual events and the related information output and the patient's reactions, e.g. in the form of a so-called feedback score, via a corresponding display.

The user interface (interface arrangement) can also be used to configure the system, for example with the following functions:
Display of a currently active addressing (messaging) of the patient,
Indication of the patient's degree of participation or involvement,
Activation or deactivation of the system,
Display or management of examination plans, treatment plans, care plans,
Import and configuration of rules for a rule-based system, e.g. event conditions, rules for suppressing instructions,
Import of data or files,
Configuration of input and output interfaces,
Configuration of input sources for data elements,
Configuration of output sources for instructions,
Configuration of the system on the patient

Embodiments can show how the support system can be configured as a multilingual support system as an example. The support of several national languages offers the advantage for both the nursing staff and the patient that any language barriers can be overcome. To output the voice instructions in different national languages, the individual instructions can already be pre-translated so that text identifiers for the respective translations can be stored in the data storage unit in each available national language and activated via a language switch. By using symbolic text identifiers, German-language announcements (here: “Atmen Sie bitte langsam ein und wieder aus”) as well as their English-language translation (“Please breathe in and out more slowly”) can be easily integrated into the support system.

The patient can be proactively prepared for upcoming situations by supporting the clinical staff's intention to act or by automated ventilation. Here are some examples of adjustments to certain ventilation parameters in noticeably larger steps for the patient:
An increase in tidal volume increases the perceived pressure in the lungs. Such a situation can be perceived as unpleasant by the patient.
With a reduction of tidal volume or a decrease in the respiratory rate, the patient may experience shortness of breath.

Even if both situations cannot be avoided, the patient can be at least partially relieved of any fears and uncertainties that may arise by making appropriate announcements using a voice output.
When initiating a ventilation maneuver for diagnostic purposes, the patient may experience higher pressures in the lungs, at least temporarily. Such maneuvers serve to maintain lung function (e.g. recruitment maneuvers), are used to determine lung parameters (e.g. low-flow maneuvers) or are required for diagnostic purposes (e.g. inspiration hold maneuvers for imaging procedures). Such situations can be perceived as unpleasant by the patient. Acoustic support for the patient to relax before and/or during the maneuvers can reduce the likelihood of interference, for example due to a lack of patient cooperation during the maneuvers, such as counter-breathing by the patient.
If a build-up of secretions in the airways has been identified, the patient may be asked to actively cough. By coughing, the secretion can be transported away naturally via the airways. This can reduce the frequency of the often stressful and unpleasant suctioning of secretions for the patient.

Embodiments can show how permanent or temporary muting of alarms can be implemented as an example. Communication, i.e. addressing (messaging) the patient, can be suspended if—for example, to prevent alarms—too many instructions have been issued in a short time, but the patient needs rest to recover. If the patient is asleep, relaxed or sedated, communication to the patient can be shifted to instructions or address at the emotional access level. Pleasant sounds or music can then be addressed or messaged to the patient instead of voice instructions.

Embodiments can show how influencing and/or guiding patients during therapy by addressing them or giving instructions can be implemented by way of example. The patient reacts to being addressed and changes their behavior, for example their breathing, in such a way that the result is an orientation towards achieving a therapy goal or a therapy corridor. The therapy corridor can be defined, for example, by range limits of respiratory physiological parameters such as respiratory rate, minute volume and oxygen saturation.

The control unit obtains information relating to the therapy target or the therapy corridor by means of trend analyses of the data elements provided via the interface.

The present invention makes it possible to avoid alarms and thus reduce avoidable alarms by addressing the patient in a targeted manner and incorporating the patient's reaction to this as feedback.

The present invention makes it possible to reduce anxiety and stress situations when waking up from anesthesia or sedation by talking to (addressing or messaging) the patient, e.g. by explaining the situations in which the patient finds themself and by making the ambient noise normal.

The present invention also makes it possible to improve the weaning of the patient from mechanical ventilation by means of targeted instructions and addressing.

The present invention enables the patient to be informed via addressing and instructions if the patient's intentions are recognized. In this way, patient endangerment (e.g. independent extubation or bed escape) can be prevented.

The present invention also makes it possible to distract the patient from their current situation in order to create a positive basic attitude, for example by addressing them with selected music, voice instructions from relatives, pleasant sounds from the private sources/environment (e.g. birdsong, the sound of the sea).

In summary, the present invention makes it possible to make the patient's stay pleasant and comfortable under the given circumstances.

In the following, exemplary embodiments of the invention are explained in more detail with reference to FIGS. 1 to 3, without limiting the generality of the inventive concept. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a basic structure of a support system for supporting a patient;

FIG. 2 is a schematic view showing the support system shown in FIG. 1 in an operational environment; and

FIG. 3 is a schematic view showing the principle function of a support system according to FIG. 1 or 2.

### DESCRIPTION OF PREFERRED EMBODIMENTS

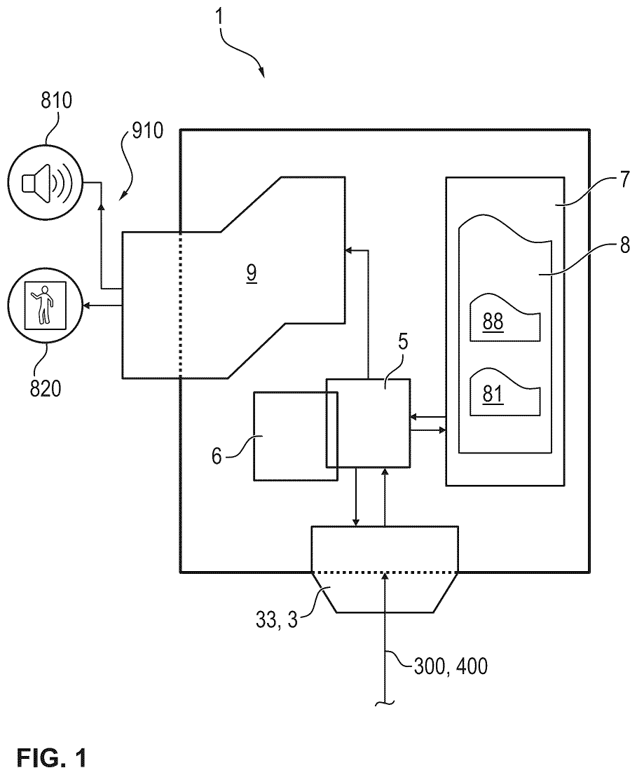

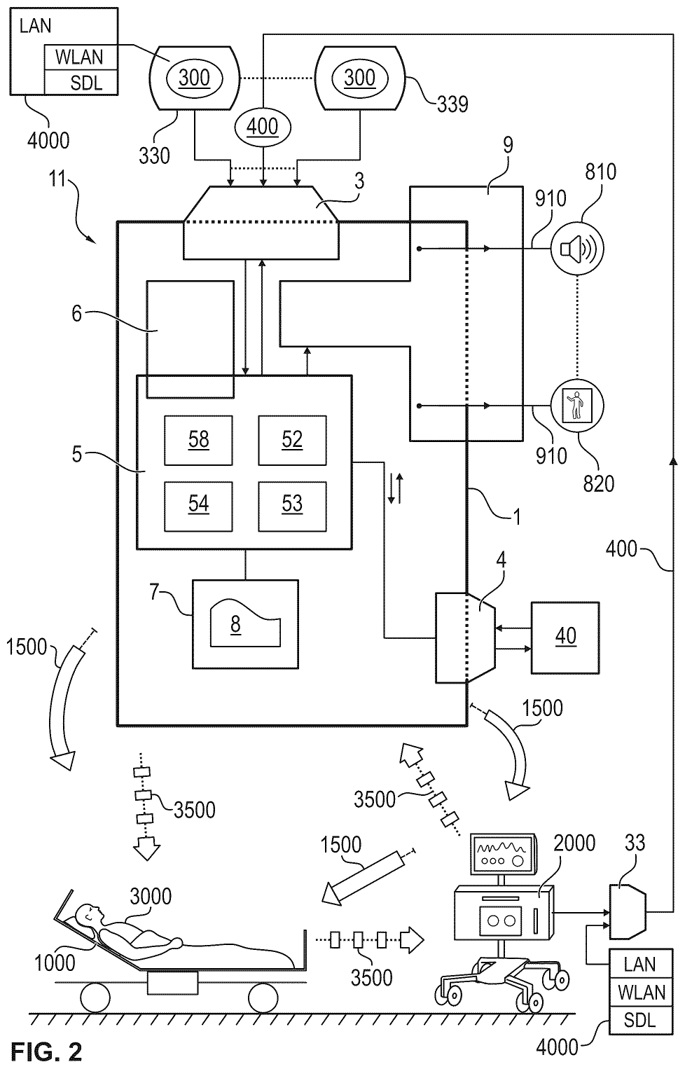

Referring to the drawings, FIG. 1 shows a support system 1 with interfaces 3, 33, a control unit 5, a data storage unit 7, an output unit 9. A data analysis unit 6 is assigned to the control unit 5, whereby the data analysis unit 6 can be configured as a sub-unit of the control unit 5 or as a separate unit. The data storage unit 7 is configured to store or deposit elements with instructions 8, instruction families 88 and/or sequences 81 with text or speech to form instructions. In this FIG. 1, exemplary means for an acoustic output 810, such as a loudspeaker system, and/or means for a visual output 820, such as a screen, are arranged on the output unit 9 for an output 910 of the instructions 8. The control unit 5 and the data analysis unit 6 are configured to analyze data elements 300 or confirmation address 400 received via the interfaces 3, 33. The data elements 300 as well as the confirmation address can indicate a variety of parameters. These include, for example, parameters of the airways, the lungs, ventilation parameters, physiological parameters, respiratory physiological parameters. They also include data elements that indicate status data, alarm situations, care situations, therapy situations, diagnostic situations, physical situations, pathological situations, which indicate psychologically induced situations or physiological situations of the patient 3000 (FIG. 2).

In particular, this also includes data elements that indicate neurological, physical parameters, a parameter of the cardiovascular, blood circulation, lymphatic or vascular system. The data elements 300 as well as the confirmation address 400 can be based on measured values, 2D or 3D image or video data or audio data.

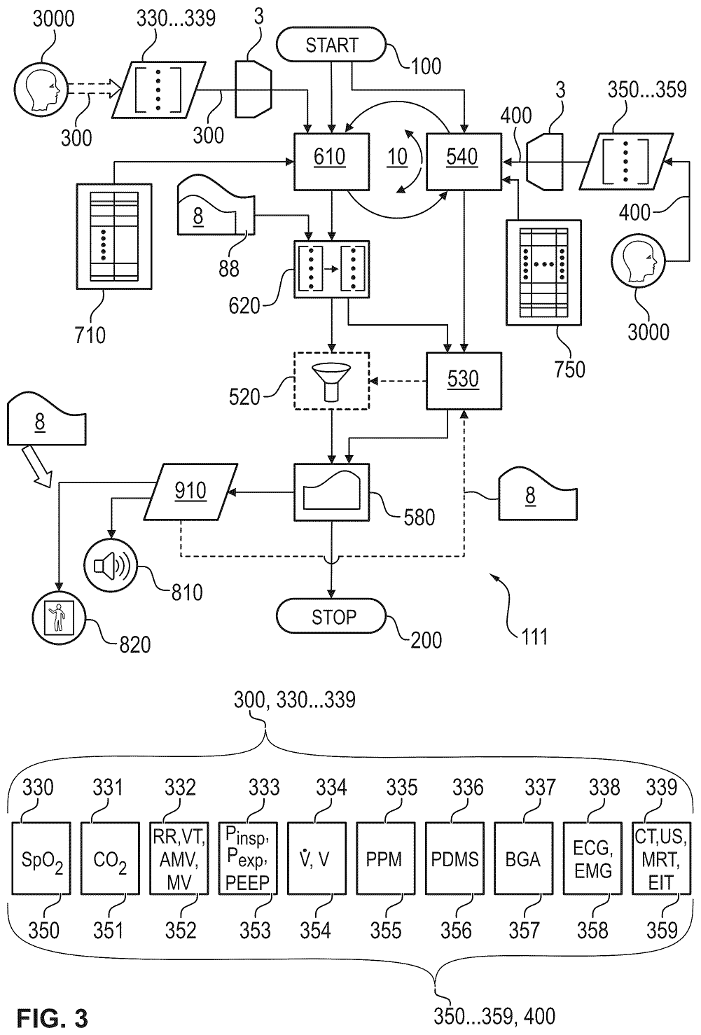

When performing the analysis of the data elements 300 and confirmation address 400, the control unit 5 and the analysis unit 6 can, for example, perform comparisons with predetermined upper and/or lower threshold values, patterns, comparison values, value corridors 710, 750 (FIG. 3) stored in the data memory 7 in order to determine events and situations or to evaluate the reaction of the patient 3000 (FIG. 2).

When performing the analysis of the confirmation address 400, the control unit 5 and the analysis unit 6 each perform analyses and comparisons with upper and/or lower threshold values, value corridors, patterns, comparison values 750 (FIG. 3) in order to determine the reactions of the patient 3000 (FIG. 3).

In FIG. 2, the support system 1 with output unit 9, interface 3, control unit 5 with associated data analysis unit 6 and data storage unit 7 according to FIG. 1 is shown in the operational environment 11 with a patient 3000. Identical elements in FIG. 1 and FIG. 2 are labeled with the same reference numbers in FIGS. 1 and 2. Patient 3000 is lying in a hospital bed 1000 and is connected to a ventilator 2000; cable, data and tube connections between patient 1000 and ventilator 2000 are not shown in this FIG. 2 in order to maintain a clear representation. FIG. 2 shows arrows indicating a support effect 1500 from support system 1 to patient 3000 in a feedback loop 3500 consisting of patient bed 1000, ventilator 2000, support system 1 and patient 3000. A filtering and prioritization unit 52, an event analysis unit 53, a reaction analysis unit 54, a selection unit 58 are shown as sub-elements with sub-functions of the control unit 5. Interactions between the sub-elements 52, 53, 54, 58 within the control unit 5 and the analysis unit 6 are not shown in detail in FIG. 2 for the sake of clarity.

Data elements 300 from various data sources 330-339 with measurement data and status data can be fed to the control unit 5 via interfaces 3.

A communication interface 4 is used to connect an input/display unit (user interface, UI, GUI) 40. The output unit 9 is provided with selected elements with instructions 8 by the control unit 5, which are stored in the data memory 7.

The output unit 9 carries out instruction outputs 910, for example as acoustic outputs 810 or visual outputs 820, to the patient 3000. A further interface 33 is shown as an example on the ventilator 2000 in this FIG. 2. This is intended to illustrate how confirmation address 400, for example in the form of measured values and/or their changes in airway and/or ventilation pressures, flow rates can be provided by the ventilator 2000 to the support system 1 via the interface 3. In addition, data 300, 400 from data networks 4000 can be provided to the support system 1 via the interfaces 3, 33.

FIG. 3 shows a schematic representation of the principle mode of operation of a support system 1 (FIG. 1) in the operational environment 11 (FIG. 2), including the reactions of a patient 3000 using a sequence 111 with functional elements 100 (START), 330 . . . 339, 350 . . . 359, 610, 540, 620, 710, 740, 530, 580, 910, 200 (STOP), input elements 300, 400 and output elements 810, 820.

The process shown can be carried out, for example, with a support system as shown in FIG. 1 using the components described in FIG. 1 or 2, such as interface 3, control unit 5, data analysis unit 6, data storage unit 7 and output unit 9, with coordination of control unit 5 (FIGS. 1, 2).

Identical elements in FIGS. 1, 2 and FIG. 3 are labeled with the same reference numbers in FIGS. 1, 2 and 3.

Beginning with a start 100, data elements 300 are imported into the sequence 111 in order to perform an event analysis 610. The data elements 300 are indexed, for example and preferably, the parameters or variables described with respect to FIGS. 1-2 and in the context of the general description of the present invention.

In this FIG. 3, the following devices or systems are shown as examples as data sources 330-339 for data elements 300: devices for measuring oxygen saturation (SpO₂) in the blood 330, devices for measuring carbon dioxide (CO₂) in the respiratory gas 331, devices for measuring respiratory parameters (RR, VT, MV, AMV) 332, devices or sensors for determining inspiratory and expiratory airway pressures (Pᵢₙₛₚ, Pₑₓₚ PEEP) 333, devices or sensors for determining inspiratory and expiratory flow rates (V′ᵢₙₛₚ, V′ₑₓₚ) 334 or volumes (Vᵢₙₛₚ, Vₑₓₚ) 334, systems for physiological patient monitoring (PPM) 335, patient data management systems (PDMS) 336, system for blood gas analysis (BGA) 337, measurement system for electrocardiography (ECG) 338 or electromyography (EMG) 338, systems for imaging (EIT, CT, MRI, ultrasonic (US)) 339.

During the event analysis 610, for example, comparisons are made with predetermined information 710 stored in the data memory 7 regarding upper and/or lower threshold values, patterns, comparison values or value corridors. The result of the event analysis 610 is then used to make an assignment 620 between the identified event with an instruction 8 or instruction family 88. This is followed by data filtering and data prioritization 520, followed by instruction selection and provision 580.

Beginning with the start 100—in addition to the recording of situations and events—confirmation address 400 are imported at the same time in the same sequence 111 in order to carry out a reaction analysis 540. The purpose of the reaction analysis 540 is to use the confirmation address 400 to check whether the previously executed instructions 8 have had an effect on the patient 3000. The confirmation address 400 represents and indicates, for example and preferably, the parameters or variables described with respect to FIGS. 1 to 2 and in the context of the general description of the present invention. In this FIG. 3, the following devices or systems are shown as examples as possible sources 350-359 of confirmation (feedback) address 400:
Devices for measuring oxygen saturation (SpO₂) in the blood 350, devices for measuring carbon dioxide (CO₂) in the respiratory gas 351, devices for measuring respiratory parameters (RR, VT, MV, AMV) 352, devices or sensors for determining inspiratory and expiratory airway pressures (Pᵢₙₛₚ, Pₑₓₚ PEEP) 353, devices or sensors for determining inspiratory and expiratory flow rates (V′ᵢₙₛₚ, V′ₑₓₚ) 354 or volumes (Vᵢₙₛₚ, Vₑₓₚ) 354,
Systems for physiological patient monitoring (PPM) 355, patient data management systems (PDMS) 356, systems for blood gas analysis (BGA) 357, measurement systems for electrocardiography (ECG) 358 or electromyography (EMG) 358, systems for imaging (EIT, CT, MRI, ultrasonic (US)) 359. During the reaction analysis 540, for example, comparisons are made with predetermined information 750 stored in the data memory 7 on upper and/or lower threshold values, patterns, comparison values or value corridors in order to classify the patient's reactions and determine the patient's degree of cooperation or willingness to participate.

The result of the reaction analysis 540 is fed to an instruction-reaction adjustment 530. The instruction-reaction comparison 530 can therefore also be referred to as a feedback comparison 530. Both the assignment 620 of an instruction family 88 or instruction 8 and the result of the reaction analysis 540 are included in the feedback comparison 530. Results from the feedback comparison 530 and the instruction assignment 620 resulting from the event analysis 610 are included in the data filtering and data prioritization 520.

Thus, the data filtering and data prioritization 520 includes, for example, situations/scenarios (constellations) of how often an instruction 8 from the same instruction family 88 was issued to the patient 3000 in a past time period, what priority the identified event 610 has on a predefined scale of priorities, and how many address with what priority and associated with defined instruction families are queued for output to an instruction selection and provision 580. The instruction selection and provision 580 causes an instruction output 910 and transmits the selected instruction 8 to the feedback comparison 530. The instruction 8 is then sent to the patient 3000 by the instruction output 910 as an acoustic instruction 810 and/or visual output 820 for addressing or messaging to the patient 3000. The sequence ends with a STOP 200.

In summary, the sequence 111 according to FIG. 3 thus forms a feedback loop (closed loop) 10 comprising data elements 300, events 610 identified therefrom, instructions 8 and confirmation address 400, wherein the confirmation address 400 are reactions (responses) of the patient 3000 to instructions 8 previously addressed or messaged to the patient.

While specific embodiments of the invention have been shown and described in detail to illustrate the application of the principles of the invention, it will be understood that the invention may be embodied otherwise without departing from such principles.

### REFERENCE NUMBER LIST

1 Support system, schematic
3 Interfaces (Ethernet, LAN, WLAN, Bluetooth, wireless, optical)
4 Communication interface
5 Control unit
6 Data analysis unit
7 Data storage unit
8 Elements with instructions
9 Output unit
10 Feedback system
11 Operating environment
33 Interface
40 Input/display unit, user interface
52 Filtering and prioritization unit
53 Event analysis unit
54 Reaction (response) analysis unit
58 Selection unit
81 Speech sequences, text sequences
88 Instruction family
100 START
111 Analysis process (flow) in the support system
200 STOP
300 Data element
330—Data input, data sources for measurement data, status data,
350—Data input, data sources for confirmation (feedback) address
400 Confirmation (feedback) address
350—Data input, data sources for patient reactions (responses)
520 Data filtering and data prioritization
530 Instruction-reaction (response) comparison (matching); instruction-feedback comparison (matching)
540 Reaction analysis
580 Instruction selection and provision (delivery)
610 Event analysis
620 Assignment of instructions, instruction assignment
710 Data memory, collection of instructions, instruction classes
750 Data memory, collection of reactions (responses), collection of confirmation (feedback) address
810 Acoustic output
820 Visual output
910 Instruction output
1000 Patient bed, lying surface
1500 Supporting effect
2000 Medical device, ventilator
3000 Patient
3500 Feedback loop
4000 Data network, LAN, WLAN, SDC, Ethernet, Bluetooth, NFC

What is claimed is:

1. A support system for assisting a patient during medical diagnostics or therapy, the support system comprising:
an interface arrangement comprising one or more interfaces configured to receive at least one data element from a group of data elements;
a data storage unit for storing instructions, or for storing groups of instructions or for storing instruction families, wherein the instructions stored in the data storage unit are configured to address at least one of the human senses including at least one of the sense of hearing, sense of sight, sense of taste, sense of smell, sense of touch or sense of feel;
an output unit; and
a control unit with an associated data analysis unit,
wherein the control unit is configured to coordinate the interface, the output unit, the data storage unit and the data analysis unit,
wherein the data analysis unit is configured to analyze the data elements and to provide an analysis result,
wherein the control unit is configured to use the analysis result to select at least one instruction from the group of instructions or instruction families stored in the data storage unit and to provide the at least one selected instruction to the output unit,
wherein the output unit is configured to address or issue the at least one selected instruction to the patient,
wherein the interface arrangement is configured to detect at least one confirmation address from a group of confirmation addresses,
wherein the at least one confirmation address indicates a reaction or feedback from the patient to the at least one instruction previously addressed to or issued to the patient or changes in the therapy or treatment environment, in a patient care situation or in the environment of the patient compared to a time before the instruction was addressed to or issued to the patient,
wherein during operation of the support system the control unit is configured to continuously select or adapt the instructions based on the data elements and the confirmation addresses and to coordinate the provision of the instructions to the output unit.

2. A support system according to claim 1,
wherein the at least one data element is configured as an element from a group of data elements and is based on measured variables or variables derived from measured variables, parameters, information, planning data, calendar data, and/or time data,
wherein the group of data elements comprises at least one of the following data elements:
data elements which indicate physical, pathological, physiological or psychological situations of the patient;
data elements which indicate a parameter of the upper airways, the lower airways or the lungs, a respiratory physiological parameter or a ventilation parameter, including pressure levels, breathing or ventilation frequencies, flow rates, volumes, compositions of inhaled or exhaled gases, wherein the data elements indicate an exceedance of the parameter of the upper airways, the lower airways or the lungs, of the respiratory physiological parameter or ventilation parameter above a predetermined threshold value for a predetermined period of time or for a predetermined number of times, or wherein the data elements indicate a shortfall of the parameter of the upper airways, the lower airways or the lungs, the respiratory physiological parameter or the ventilation parameter below a predetermined threshold value for a predetermined period of time or for a predetermined number of times;
data elements which indicate a neurological parameter, wherein the data elements indicate an exceeding of the neurological parameter above a predetermined threshold value for a predetermined period of time or for a predetermined number of times, or wherein the data elements indicate a falling below of the neurological parameter below a predetermined threshold value for a predetermined period of time or for a predetermined number of times;
data elements which indicate a physical parameter, wherein the data elements indicate an exceeding of the physical parameter above a predetermined threshold value for a predetermined period of time or for a predetermined number of times, or wherein the data elements indicate a falling below of the physical parameter below a predetermined threshold value for a predetermined period of time or for a predetermined number of times;
data elements which indicate a parameter of the cardiovascular system or of the blood circulation, vascular or lymphatic system, including indicating pressure levels or flow or volume situations, wherein the data elements indicate an exceedance of the parameter of the cardiovascular system or of the blood circulation, vascular or lymphatic system above a predetermined threshold value for a predetermined period of time or for a predetermined number of times, or wherein the data elements indicate that the parameter of the cardiovascular system falls below a predetermined threshold value for a predetermined period of time or for a predetermined number of times;
data elements characterizing a physiologically or psychologically induced situation of pain, anxiety or panic, based on an increase in the heart rate within a predetermined time interval above a predetermined threshold value, an increase in skin moisture within a predetermined time interval above a predetermined threshold value, an increase in respiratory rate within a predetermined time interval above a predetermined threshold value, a change in muscle tone, an increase in systolic or diastolic blood pressure;
data elements which indicate alarm situations during medical therapies, diagnosis, care measures or during a regular clinical daily routine;
data elements which indicate situations with activities of the patient in the patient's environment or movement events in connection with the patient in a therapy or treatment environment or in patient care situations;
data elements which indicate situations or events in a therapy or treatment environment of the patient during the performance of medical therapies, diagnoses, care measures or the regular routine in the clinical daily routine;
data elements which are based on two-dimensional or three-dimensional image or video data and indicate status data of the patient or events in relation to the patient's environment;
data elements which are based on two-dimensional or three-dimensional image or video data and indicate situations of potential changes in location of the patient;
data elements which are based on two-dimensional or three-dimensional image or video data and indicate situations of potential disconnections of the patient from patient connection lines or patient access lines;
data elements which indicate status data or alarms of other patients or events; and
data elements which indicate status data or alarm situations of the patient or events in relation to the patient, the data elements being based on audio data including alarm signals from a direct therapy or treatment environment or a spatially close environment of the patient.

3. A support system according to claim 1, wherein the group of instructions stored in the data storage unit contains or comprises at least one of the following instructions: intentions, text modules, sentence modules, sentence fragments, speech sequences, words, syllables, audio sequences, speech cues, sound reproductions, music reproductions, sound reproductions, sound reproductions, text representations, graphic representations, visualizations, film or video sequences, individual images, individual image sequences, diagrams, symbols, pictograms.

4. A support system according to claim 1,
wherein the at least one confirmation address is formed as an element from the group of confirmation addresses and is based on mental, physical, physical, neurological, physiological or metrologically determined data or information,
wherein the group of confirmation addresses includes or comprises at least one of the following confirmation addresses:
confirmation addresses which indicate physical, pathological, physiological or psychological situations of the patient;
confirmation addresses which indicate an upper airway, lower airway or lung parameter, a respiratory physiology parameter or a ventilation parameter relative to a time prior to issuing the instruction to the patient;
confirmation addresses which indicate a parameter of the lungs, the cardiovascular system or a parameter of the blood circulation, vascular or lymphatic system including heart rate, heart rate variability, systolic blood pressure, diastolic blood pressure, wherein the confirmation addresses indicate a change in the cardiovascular system parameter or the circulatory or vascular system parameter from a time prior to the issuance of the instruction to the patient;
confirmation addresses which indicate physiologically or psychologically induced situations by a change in heart rate, skin hydration, respiratory rate or muscle tone compared to a time before the instruction was issued to the patient;
confirmation addresses which indicate events or situations, status data during medical therapies, diagnoses, care measures or during a regular routine, in a clinical daily routine,
confirmation addresses which indicate situations with movement events in a therapy or treatment environment, in the care situation or in the patient's environment;
confirmation addresses which indicate situations with activities in the patient's environment, movement events in connection with the patient in the therapy or treatment environment or in care situations;
confirmation addresses which are based on two-dimensional or three-dimensional image or video data and indicate situations with respect to the status of patient connection lines or patient access lines with respect to a point in time before the instruction is issued to the patient,
confirmation addresses which indicate situations based on two-dimensional or three-dimensional image or video data of potential changes in location of the patient relative to a time prior to the issuance of the instruction to the patient; and
confirmation addresses which indicate changes in alarm situations compared to a point in time before the instruction was issued to the patient.

5. A support system according to claim 1, wherein the control unit is configured to assign a prioritization to the instructions and to configure the temporal sequence of issuing instructions to the patient on the basis of the prioritization.

6. A support system according to claim 1, wherein the control unit is configured to determine an effect of the instructions based on instruction information and to classify the instruction or classify an effect of the instructions and to configure the output of further instructions to the patient based on the classification.

7. A support system according to claim 1, wherein the confirmation addresses are based on two-dimensional or three-dimensional image or video data or on audio data, which includes at least one of
a patient's mood state based on image or video data comprising a facial expression, facial expression or physiognomy of the patient;
a patient's mood state based on audio data, which includes sounds or sounds produced by the patient that indicate psychological or physiological situations including anxiety or panic;
a spatial situation of the patient based on two-dimensional or three-dimensional image or video data, which comprise an image of the patient in their treatment environment, in the care situation or the therapy environment.

8. A support system according to claim 1,
wherein the interface arrangement receives the at least one data element and/or the at least one confirmation address from a medical device, a diagnostic device, a therapy device, a ventilator or an anesthesia device, a medical measuring device, a physiological patient monitor, a blood gas analyzer, an electroimpedance tomography device, an ultrasound imaging device an X-ray device, a mammography device, a heart sound contraction recorder, a computer tomography device, a magnetic resonance imaging device, an imaging or camera system, a thermometer for recording body temperatures, devices for measuring carbon dioxide saturation or oxygen saturation in the blood, devices for determining the carbon dioxide concentration in respiratory gases, including a capnometer or oxy-capnometer, a heat therapy device, including a warming bed or incubator, or
wherein the support system is provided as a part or as a component of a medical device, a diagnostic device, a therapy device, a ventilator or an anesthesia device, a medical measuring device, a physiological patient monitor, an electroimpedance tomography device, an ultrasound imaging device an X-ray device, a mammography device, a computer tomography device, a magnetic resonance imaging device, an imaging or camera system, a thermometer for detecting body temperatures, a device for measuring oxygen saturation or carbon dioxide concentration in respiratory gases, including a capnometer or oxy-capnometer, a heat therapy device, including a warming bed or incubator.

9. A support system according to claim 1,
wherein the control unit is configured to continuously select the instructions based on the data elements and/or based on the confirmation addresses during operation of the support system, generating, filtering or adapting the instructions,
wherein the control unit is configured to include in the selection, generation, filtering or adaptation of the instructions at least one patient-specific criterion from a group of patient-specific criteria comprising:
name, nationality, mother tongue, gender, sexual orientation, religion;
language, mother tongue, dialect, sign language;
age, height, weight, body mass index;
mood, state of mind;
clinical picture, diagnosis, medication, anesthesia;
therapy situation, care situation, degree of therapy, degree of care, duration of therapy;
physical, pathological, physiological or psychological situations of the patient;
type and balance of diet related to fats and carbohydrates;
dietary arrangements including special diets;
information from a nutrition and fluid management system;
length of stay in a therapy or treatment environment; and
privacy in the therapy or treatment environment.

10. A process for supporting a patient during a performance of medical treatments, therapies, and diagnoses, the process comprising the steps of:
providing a support system for assisting a patient during medical diagnostics or therapy, the support system comprising: an interface arrangement comprising one or more interfaces configured to receive at least one data element from a group of data elements; a data storage unit for storing instructions, or for storing groups of instructions or for storing instruction families, wherein the instructions stored in the data storage unit are configured to address at least one of the human senses, including at least one of the sense of hearing, sense of sight, sense of taste, sense of smell, sense of touch or sense of feel; an output unit; and a control unit with an associated data analysis unit, wherein the control unit is configured to coordinate the interface, the output unit, the data storage unit and the data analysis unit, wherein the data analysis unit is configured to analyze the data elements and to provide an analysis result, wherein the control unit is configured to use the analysis result to select at least one instruction from the group of instructions or instruction families stored in the data storage unit and to provide the at least one selected instruction to the output unit, wherein the output unit is configured to address or issue the at least one selected instruction to the patient, wherein the interface arrangement is configured to detect at least one confirmation address from a group of confirmation addresses, wherein the at least one confirmation address indicates a reaction or feedback from the patient to the at least one instruction previously addressed to or issued to the patient or changes in the therapy or treatment environment, in a patient care situation or in the environment of the patient compared to a time before the instruction was addressed to or issued to the patient, wherein during operation of the support system the control unit is configured to continuously select or adapt the instructions based on the data elements and the confirmation addresses and to coordinate the provision of the instructions to the output unit;
performing a continuously repeating sequence of steps comprising:
reading in at least one data element from a group of data elements;
evaluating the data elements with regard to situations or events that are related to a condition or behavior of the patient;
selecting an instruction to the patient from the group of instructions or the families of instructions;
issuing the instruction to the patient;
reading in at least one confirmation address from a group of confirmation addresses; and
evaluating the confirmation address with regard to situations or events that are related to a condition or behavior of the patient.

11. A process according to claim 10,
wherein the at least one data element is configured as an element from a group of data elements and is based on measured variables or variables derived from measured variables, parameters, information, planning data, calendar data, and/or time data,
wherein the group of data elements comprises at least one of the following data elements:
data elements which indicate physical, pathological, physiological or psychological situations of the patient;
data elements which indicate a parameter of the upper airways, the lower airways or the lungs, a respiratory physiological parameter or a ventilation parameter, including pressure levels, breathing or ventilation frequencies, flow rates, volumes, compositions of inhaled or exhaled gases, wherein the data elements indicate an exceedance of the parameter of the upper airways, the lower airways or the lungs, of the respiratory physiological parameter or ventilation parameter above a predetermined threshold value for a predetermined period of time or for a predetermined number of times, or wherein the data elements indicate a shortfall of the parameter of the upper airways, the lower airways or the lungs, the respiratory physiological parameter or the ventilation parameter below a predetermined threshold value for a predetermined period of time or for a predetermined number of times;
data elements which indicate a neurological parameter, wherein the data elements indicate an exceeding of the neurological parameter above a predetermined threshold value for a predetermined period of time or for a predetermined number of times, or wherein the data elements indicate a falling below of the neurological parameter below a predetermined threshold value for a predetermined period of time or for a predetermined number of times;
data elements which indicate a physical parameter, wherein the data elements indicate an exceeding of the physical parameter above a predetermined threshold value for a predetermined period of time or for a predetermined number of times, or wherein the data elements indicate a falling below of the physical parameter below a predetermined threshold value for a predetermined period of time or for a predetermined number of times;
data elements which indicate a parameter of the cardiovascular system or of the blood circulation, vascular or lymphatic system, including indicating pressure levels or flow or volume situations, wherein the data elements indicate an exceedance of the parameter of the cardiovascular system or of the blood circulation, vascular or lymphatic system above a predetermined threshold value for a predetermined period of time or for a predetermined number of times, or wherein the data elements indicate that the parameter of the cardiovascular system falls below a predetermined threshold value for a predetermined period of time or for a predetermined number of times;
data elements characterizing a physiologically or psychologically induced situation of pain, anxiety or panic, based on an increase in the heart rate within a predetermined time interval above a predetermined threshold value, an increase in skin moisture within a predetermined time interval above a predetermined threshold value, an increase in respiratory rate within a predetermined time interval above a predetermined threshold value, a change in muscle tone, an increase in systolic or diastolic blood pressure;
data elements which indicate alarm situations during medical therapies, diagnosis, care measures or during a regular clinical daily routine;
data elements which indicate situations with activities of the patient in the patient's environment or movement events in connection with the patient in a therapy or treatment environment or in patient care situations;
data elements which indicate situations or events in a therapy or treatment environment of the patient during the performance of medical therapies, diagnoses, care measures or the regular routine in the clinical daily routine;
data elements which are based on two-dimensional or three-dimensional image or video data and indicate status data of the patient or events in relation to the patient's environment;
data elements which are based on two-dimensional or three-dimensional image or video data and indicate situations of potential changes in location of the patient,
data elements which are based on two-dimensional or three-dimensional image or video data and indicate situations of potential disconnections of the patient from patient connection lines or patient access lines;
data elements which indicate status data or alarms of other patients or events; and
data elements which indicate status data or alarm situations of the patient or events in relation to the patient, the data elements being based on audio data including alarm signals from a direct therapy or treatment environment or a spatially close environment of the patient.

12. A process according to claim 10, wherein the group of instructions stored in the data storage unit contains or comprises at least one of the following instructions: intentions, text modules, sentence modules, sentence fragments, speech sequences, words, syllables, audio sequences, speech cues, sound reproductions, music reproductions, sound reproductions, sound reproductions, text representations, graphic representations, visualizations, film or video sequences, individual images, individual image sequences, diagrams, symbols, pictograms.

13. A process according to claim 10,
wherein the at least one confirmation address is formed as an element from the group of confirmation addresses and is based on mental, physical, physical, neurological, physiological or metrologically determined data or information,
wherein the group of confirmation addresses includes or comprises at least one of the following confirmation addresses:
confirmation addresses which indicate physical, pathological, physiological or psychological situations of the patient;
confirmation addresses which indicate an upper airway, lower airway or lung parameter, a respiratory physiology parameter or a ventilation parameter relative to a time prior to issuing the instruction to the patient;
confirmation addresses which indicate a parameter of the lungs, the cardiovascular system or a parameter of the blood circulation, vascular or lymphatic system including heart rate, heart rate variability, systolic blood pressure, diastolic blood pressure,
wherein the confirmation addresses indicate a change in the cardiovascular system parameter or the circulatory or vascular system parameter from a time prior to the issuance of the instruction to the patient,
confirmation addresses which indicate physiologically or psychologically induced situations by a change in heart rate, skin hydration, respiratory rate or muscle tone compared to a time before the instruction was issued to the patient,
confirmation addresses which indicate events or situations, status data during medical therapies, diagnoses, care measures or during the regular routine in a clinical daily routine, confirmation addresses which indicate situations with movement events in a therapy or treatment environment, in a care situation or in the patient's environment,
confirmation addresses which indicate situations with activities in the patient's environment, movement events in connection with the patient in the therapy or treatment environment or in care situations,
confirmation addresses which are based on two-dimensional or three-dimensional image or video data and indicate situations with respect to the status of patient connection lines or patient access lines with respect to a point in time before the instruction is issued to the patient,
confirmation addresses which indicate situations based on two-dimensional or three-dimensional image or video data of potential changes in location of the patient relative to a time prior to the issuance of the instruction to the patient,
confirmation addresses which indicate changes in alarm situations compared to a point in time before the instruction was issued to the patient.

14. A process according to claim 10, further comprising providing a computer program or computer program product provided with a tangible non-transitory computer-readable medium comprising a program code for performing one or more of the repeating sequence of process steps.

15. A computer program or computer program product provided with a tangible, non-transitory computer-readable medium comprising a program code for supporting a patient during a performance of medical treatments, therapies, diagnoses, with a support system for assisting a patient during medical diagnostics or therapy, the support system comprising: an interface arrangement comprising one or more interfaces configured to receive at least one data element from a group of data elements; a data storage unit for storing instructions, or for storing groups of instructions or for storing instruction families, wherein the instructions stored in the data storage unit are configured to address at least one of the human senses, including at least one of the sense of hearing, sense of sight, sense of taste, sense of smell, sense of touch or sense of feel; an output unit; and a control unit with an associated data analysis unit, wherein the control unit is configured to coordinate the interface, the output unit, the data storage unit and the data analysis unit, wherein the data analysis unit is configured to analyze the data elements and to provide an analysis result, wherein the control unit is configured to use the analysis result to select at least one instruction from the group of instructions or instruction families stored in the data storage unit and to provide the at least one selected instruction to the output unit, wherein the output unit is configured to address or issue the at least one selected instruction to the patient, wherein the interface arrangement is configured to detect at least one confirmation address from a group of confirmation addresses, wherein the at least one confirmation address indicates a reaction or feedback from the patient to the at least one instruction previously addressed to or issued to the patient or changes in the therapy or treatment environment, in a patient care situation or in the environment of the patient compared to a time before the instruction was addressed to or issued to the patient, wherein during operation of the support system the control unit is configured to continuously select or adapt the instructions based on the data elements and the confirmation addresses and to coordinate the provision of the instructions to the output unit, the program code being be executed on a computer, a processor, or a programmable hardware component for performing one or more of the processes steps of:
performing a continuously repeating sequence of steps comprising:
reading in at least one data element from a group of data elements;
evaluating the data elements with regard to situations or events that are related to a condition or behavior of the patient;
selecting an instruction to the patient from the group of instructions or the families of instructions;
issuing the instruction to the patient;
reading in at least one confirmation address from a group of confirmation addresses; and
evaluating the confirmation address with regard to situations or events that are related to a condition or behavior of the patient.
